(19) United States
(12) Patent Application Publication
(10) Pub. No.: US 2004/0068273 A1
(43) Pub. Date: Apr. 8, 2004

(54) Automatic laparoscopic incision closing apparatus

(75) Inventors: Bruce L. Fariss, Knoxville, TN (US); Ivan N. Cooper, Knoxville, TN (US)

Correspondence Address: PITTS AND BRITTIAN P C, P O BOX 51295, KNOXVILLE, TN, 37950-1295, US

(73) Assignee: Ibionics Corporation, Knoxville, TN

(21) Appl. No.: 10/263,106

(22) Filed: Oct. 2, 2002

(51) Int. Cl. A61B 17/04

(52) U.S. Cl. 606/144

(57) ABSTRACT

An incision closing apparatus for simultaneous insertion of suture needles through adjacent sides of an incision in a fascia layer within a patient. An insertion end is sized for insertion through the incision and a shield is retractably extended from the insertion end positioned interior of the fascia layer. A plurality of elongated spokes having needles thereon are extended from openings within the insertion end for ejection of needles having attached suture filaments through adjacent sides of the fascia layer incision. Each needle is imbedded into the extended shield to retain each needle from piercing an internal organ. Upon retraction of the shield into the insertion end, with each needle imbedded in the shield, the insertion end is removed from the incision, leaving suture filaments inserted through adjacent sides of the fascia layer incision for closure of the incision within the patient.

### CROSS REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

1. Field of Invention

This invention pertains to devices for positioning sutures to close an incision within a patient. More particularly, this invention pertains to an apparatus and a method for positioning sutures through sides of an internal incision through adjacent tissue layers within a patient.

2. Description of the Related Art

Prior laparoscopic surgical procedures utilize a laparoscope for abdominal exploration, for repair of internal organ defects, and for excision of tissue. The laparoscope and accompanying cannula and tracer are inserted through small incisions in the outer, dermal layer and an internal fascia layer of about four to about twenty millimeters in length. Upon removal of the laparoscope, rapid closure of an internal incision through a fascia layer within the abdomen is important to allow the patient to begin post-operative recovery. Prior procedures for closure of internal incisions are typically tedious to accomplish and lengthy in time. Prior laparoscopic surgical procedures for closure of internal incisions typically require insertion with long-handled devices of one needle and attached suture threads in a stitching pattern for closure of internal incisions. An alternative surgical procedure includes manipulation of a medical stapler or needle insertion device for placement of numerous staples or sutures, one at a time, along respective first and second sides of the internal incision. If the internal sutures are not positioned securely along the adjacent walls of the incision due to the limited maneuvering space inside the patient, and/or movement of fat tissue above the internal incision, the sutures may pull out of the fascia layer with a resulting need for additional surgery.

Typical prior art suture devices for closure of internal incisions are illustrated in FIGS. 1a-1b, 2a-2b and 3. As illustrated in FIGS. 1a and 1b, one prior device 40 includes an elongated member 42 having a distal, insertion end 44 having a pivotable needle end 46 for insertion of a suture thread or wire threaded through a dermal layer of a patient's skin surface. Pivoting of the needle end 46 is controlled by a handle end 48. The prior device 40 is utilized for insertion, removal and reinsertion of the one needle on needle end 46, in a repetitive sequence of stitching movements, with the needle end 46 having a thread or wire attached for closure of an incision through the patient's dermal layer 14.

As illustrated in FIGS. 2a and 2b, an additional prior art suture device 50 includes an elongated member 52 having a slidable segment 54 attached to a cover 54′ attached to a distal end 56 for removably covering one needle end 58. A handle 60 includes a control mechanism for maneuvering a slidable segment 54 in a sliding motion 62 that moves cover 54′ toward distal end 56, therefore exposing needle end 58. Upon insertion of distal end 56 into an incision in a dermal layer 14, the handle 60 and control mechanism are manipulated to expose needle end 58, onto which a suture is attached for stitching the sides of an incision together. The one needle end 58 is manipulated by a surgeon for repeated stitching movements through the adjacent sides of the incision. The repeated stitching movements with the suture device 50 require additional surgical tools to grasp, guide, and tie-off the suture material during the stitching process. Upon completion of the stitching process, the slidable segment 54 is slidably returned 64 toward the handle 60 for movement 64 of cover 54′ to cover needle end 58 to allow removal of 50 from the patient without the needle end hooking additional tissue layers upon removal. The prior device 50 requires the steps of insertion, removal and reinsertion of the needle end 58 having a thread attached for closure of the incision in the patient.

As illustrated in FIG. 3, an additional example of a prior art device 70 includes a laparoscopic incision closure device having an elongated needle 72 having a hollow needle end 74 which is inserted through a dermal layer 14 and/or through a segment of fascia tissue 18 proximal to an incision opening through the dermal layer 14 and the fascia layer 18. A T-bar needle 76 is ejected from the hollow needle end 74 by a mechanical means for placement of one T-bar needle 76 through a segment of tissue proximal to an incision. Use of the laparoscopic incision closure device 70 requires a surgeon to insert the hollow end 74 through a first side of an incision through a fascia layer 18, manipulate the controls in the handle 78 with ejection of one T-bar needle 76 through the first side of the incision. The surgeon must then remove the device 70, reposition the hollow needle end 74 on a second side of an incision through the fascia layer 18, reinsert the needle end 74, manipulate the controls in the handle 78 with resulting ejection of a second T-bar needle 76 through the second side of an incision through the fascia layer 18. For placement of additional T-bar needles, the surgeon must remove, reposition, and reinsert the device 70 through a dermal incision previously sized for insertion of the laparoscopic device. With repetitive placement, removal, repositioning and reinsertion of the device 70 for comprehensive suturing of a deep interior incision through a fascia layer 18, the surgeon may be required to widen the dermal incision to adequately suture each side of the interior incision. Further, the surgeon must provide visual verification of proper placement of each needle and suture on adjacent sides of the incision through the fascia layer 18, requiring insertion of additional clamps and/or optic-fiber cables or a laparoscopic camera for concurrent viewing of placement of each needle and suture in order to confirm that an internal organ has not been pierced within the abdominal cavity 24. The above method of use for a laparoscopic incision closure device 70 is tedious and requires repositioning for each placement of a suture as needed to complete the incision closure procedure.

For obese patients, the procedure for closure of an internal incision through a fascia layer 18 is more difficult due to the layer of fat 16 between an incision in the outer, dermal layer 14, and the internal incision through the fascia layer 18 and adjacent layers 20, 22. After removal of the laparoscope and accompanying cannula, trocar sleeve, and/or clamps, the incision through the dermal layer may not remain aligned with the internal incision through the fascia layer 18 due to shifting of the layer of fat above the internal incision, or encroachment 26 by the fat tissue 16 (see FIG. 4). Further, a surgeon must navigate a path through the layer of fat, for each insertion, retraction, and reinsertion of the prior art needle and suture insertion devices for complete closure of an internal incision.

There is a need for a laparoscopic tool for closing of an internal incision within a patient without the requirement for repetitive insertion, retraction, and reinsertion steps for closure of an internal incision. Further, there is a need for a laparoscopic incision closing tool for simultaneous placement of a plurality of needles and suture threads through opposed sides of an internal incision with a minimum of steps for completion of the procedure.

### BRIEF SUMMARY OF THE INVENTION

According to one embodiment of the present invention, a laparoscopic incision closing apparatus is provided for generally simultaneous ejection of a plurality of suture needles for closure of an incision through internal tissue layers within a patient. The apparatus includes a housing sized to be manipulated by an operator. The housing includes a control means thereon, and a supply of suture filaments attachable thereto. An insertion member is connected to the housing, and includes an insertion end sized to be extended through an incision in a dermal layer for positioning the insertion end through an incision in one or more internal tissue layers within the patient. The insertion member includes an internal space for positioning of a first mechanical connector and a second mechanical connector along a lengthwise axis of the insertion member. A plurality of openings are disposed a spaced apart distance proximal to the distal end of the insertion end. The openings include angled channels therein that direct each one of a plurality of suture needles positioned therein on respective insertion paths through opposed sides of the incision through one or more internal tissue layers. A plurality of suture filaments are extended from the supply of suture filaments, through the insertion member, and through the insertion end for connection with each respective suture needle. The insertion end includes a shield disposed in a folded configuration within an opening in the distal end of the insertion end. The shield is extendable from the distal end by the first mechanical connector disposed to retractably extend through the insertion end. The opened shield is extended laterally from the insertion end to an extended position around the insertion end. The opened shield is positioned by the operator against an interior side of the incision through a fascia layer and adjacent tissue layers within the patient. The opened shield is disposed to receive each ejected suture needle for imbedding into a surface of the opened shield in order to retain the suture needles and to protect an internal organ from being pierced by the suture needles.

Upon simultaneous insertion of the needles through opposed sides of the incision through the fascia layer and adjacent tissue layers, and after imbedding of the needles into the opened shield, the insertion end is maneuvered inwardly within the patient for an adequate depth to allow unhindered collapsing of the opened shield while retaining the plurality of needles imbedded on the shield. The shield is collapsed by inverting into the insertion end upon re-manipulation by the operator of the control means connected to the first mechanical connector. The insertion end, having the inverted shield and imbedded needles therein, is removed from the dermal incision, trailing a plurality of suture filaments extended from each respective needle and looping through the respective sides of the incision through the fascia layer and adjacent tissue layers. The suture filaments remain retained through the fascia layer and adjacent tissue layers, and are collected by the operator for securing of the suture filaments, thereby closing the internal incision through the fascia layer and adjacent tissue layers. An efficient method for closure of an internal incision is also disclosed including a step of insertion of the incision closing apparatus, a step of extension of a shield inwardly within the patient, a step of ejection of needles and suture filaments through adjacent sides of the internal incision, and a step of retraction of the incision closing apparatus. The apparatus and method for closure provides protection of internal organs from unintended piercing by suture needles.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1a is a side view of a prior art suture device having a pivotably exposed needle for suturing an incision;

FIG. 1b is a side view of the prior art suture device of FIG. 1a, illustrating the needle pivoted into the suture device;

FIG. 2a is a side view of a prior art suture device with a slidably exposed suture needle;

FIG. 2b is a side view of FIG. 2a, illustrating the needle covered by a slidable member;

FIG. 3 is a side view of a prior art laparoscopic incision closure device illustrating placement of one T-bar suture on one side of an incision;

FIG. 4 is a side view of an incision closing apparatus of the present invention, illustrating an insertion end inserted through a dermal layer and a shield opened proximal to an internal organ within a patient;

FIG. 5 is a side perspective view of the incision closing apparatus of FIG. 4, illustrating extension of the needles toward a shield disposed in an extended position;

FIG. 6a is a side view of FIG. 5, illustrating insertion of the insertion end through an incision in a dermal layer;

FIG. 6b is a side view of FIG. 6a, illustrating the shield extended and positioned against the internal surface of an interior tissue layer;

FIG. 6c is a side view of FIG. 6b, illustrating substantially simultaneous ejection and insertion of a plurality of needles through adjacent sides of the incision through interior tissue layers with the needles imbedded into the opened shield;

FIG. 6d is a side view of FIG. 6c, illustrating the shield having the needles imbedded thereon, with suture filaments attached to the needles, and the needle spokes retracted within the insertion end;

FIG. 6e is a side view of FIG. 6d, illustrating the shield folded into a retracted position toward the insertion end with the suture filaments extended through the adjacent sides of the interior tissue layers and the needles imbedded in the shield in the retracted position;

FIG. 6f is a side view of FIG. 6e, illustrating the insertion end removed from the incision, with the retracted shield within the insertion end, and the attached suture filaments looped through the adjacent sides of the interior incision within the patient;

FIG. 7 is an exploded perspective view of FIG. 5, illustrating a needle assembly with the enclosing insertion end removed; and

FIG. 8 is a cross-section top view along 8-8 of FIG. 5, illustrating the arraignment of the needle assembly within the insertion end.

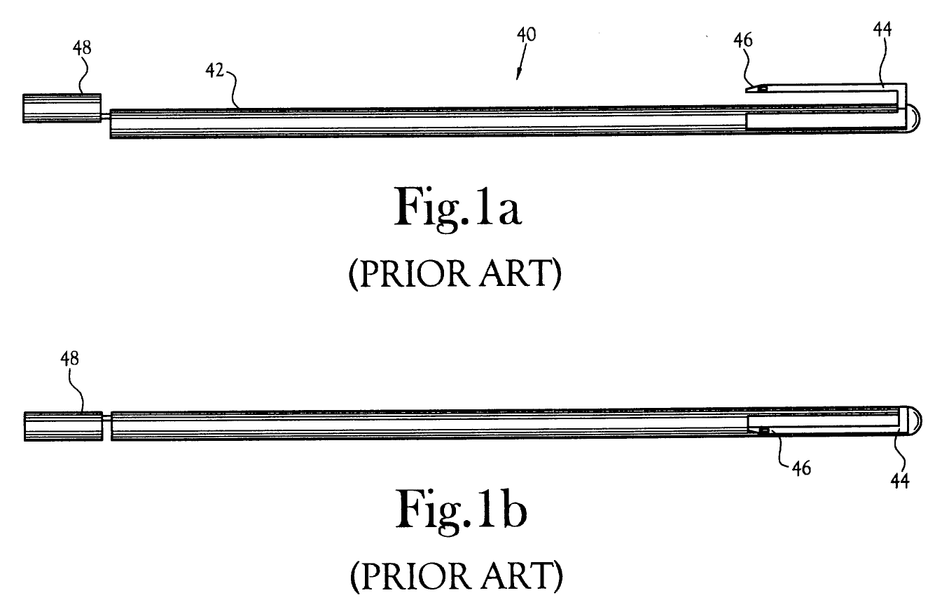

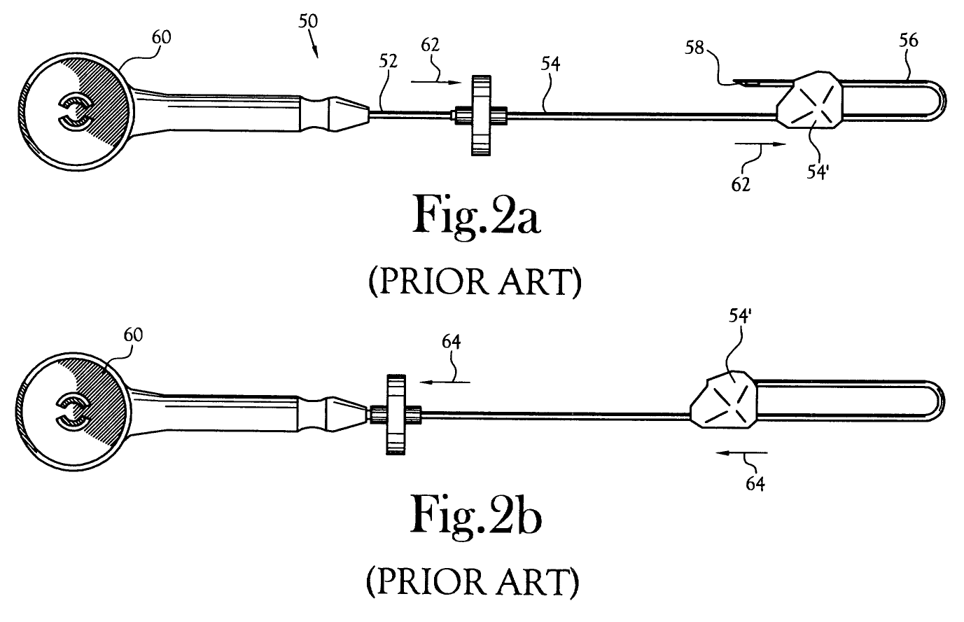

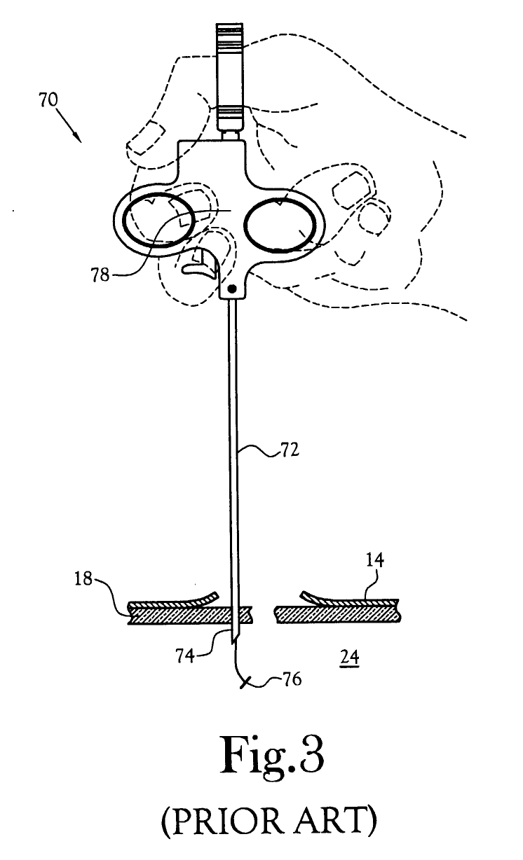

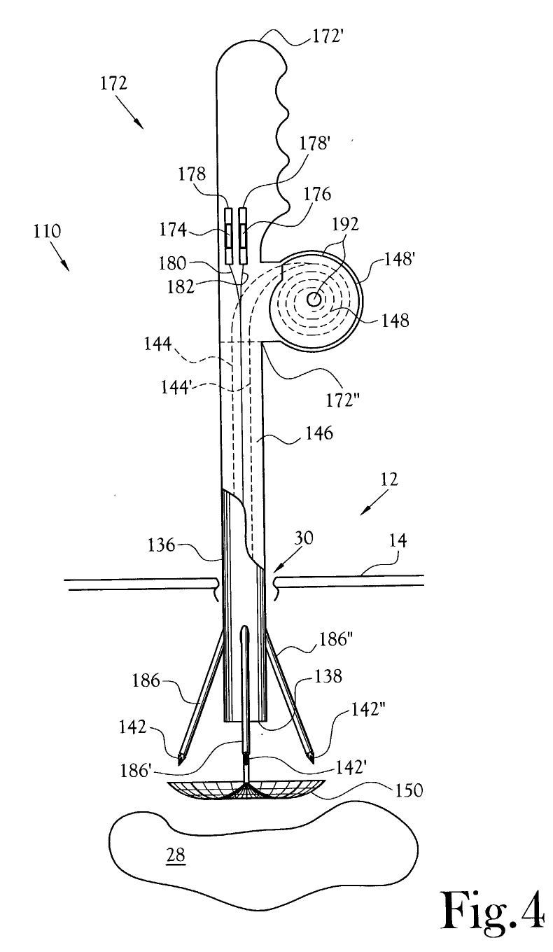

### DETAILED DESCRIPTION OF THE INVENTION

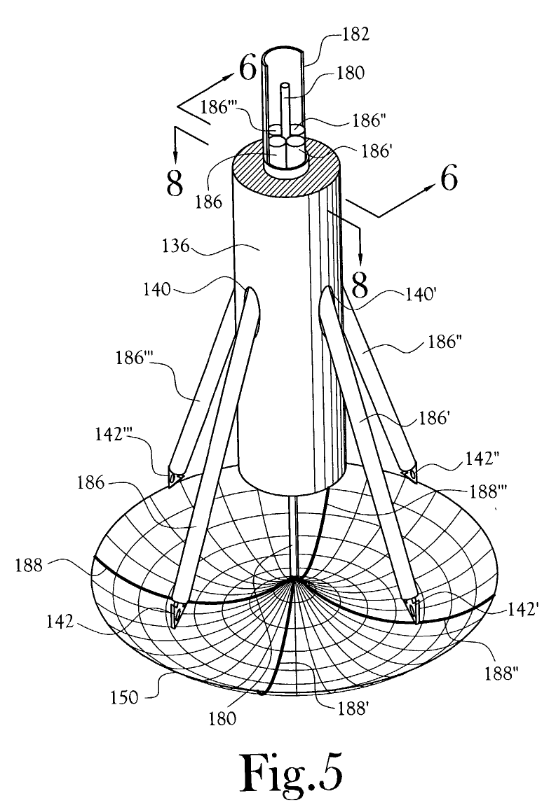

An incision closure apparatus for closing of an internal incision is disclosed as illustrated generally as a laparoscopic incision closing apparatus 110 in FIGS. 4-9b. The incision closing apparatus 110 is composed of materials that are sterilized in preparation for use in a sterile setting, or is contained in a sterile package (not shown) for use in a sterile setting such as an operating room. The incision closing apparatus 110 is utilized by a surgeon as part of an incision closing procedure after laparoscopy and/or laparotomy procedures, which are surgical procedures requiring incisions through the abdominal region 12 of a patient. Incisions in the abdominal region 12 typically include a dermal incision 30 through a patient's dermal layer 14, and an internal incision through a fascia layer 18. For a significant population of obese patients, a layer of fat 16 of substantial thickness may separate the dermal layer 14 and the fascia layer 18. A layer of muscle 20 may be disposed interior and/or exterior of the fascia layer 18, with a peritoneum layer 22 disposed interior of the fascia layer 18 and muscle layer 20. Interior of the peritoneum layer 22 is the abdominal cavity 24, in which a plurality of internal organs 28 are located, including the stomach, small and large intestines, liver, gallbladder, spleen, pancreas, kidney and ureter (separate organs not shown). When a laparoscopic procedure is attempted, a surgeon prepares a dermal incision 30 and an incision through the fascia layer 18 having an incision width 32 in order to provide an incision path through the fat layer 16 between the dermal layer 14 and the fascia layer 18. The surgeon maintains an open incision path by insertion of a cannula such as a trocar sleeve for insertion therethrough of one or more surgical tools including a laparoscope. For obese patients, the fat layer 16 can shift with the lateral shifting 26 of fat tissue into the incision path, resulting in partial or complete obstruction of the incision path below the fat layer 16. Upon completion of the surgical procedure, such as a laparoscopy and/or a laparotomy procedure, an incision closing procedure includes suturing the opposed sides and/or layers of the interior incision through the fascia layer 18 without enlarging the dermal incision 30, and without inserting a needle into one of the internal organs 28. The surgeon may prefer to suture the incision in the fascia layer 18 without excessively widening the dermal incision 30. The incision closing apparatus 110 allows the surgeon to insert and manipulate the closing apparatus 110 within a narrow incision width 32 that may be partially obstructed due to lateral shifting 26 of fat tissue during the surgical procedures.

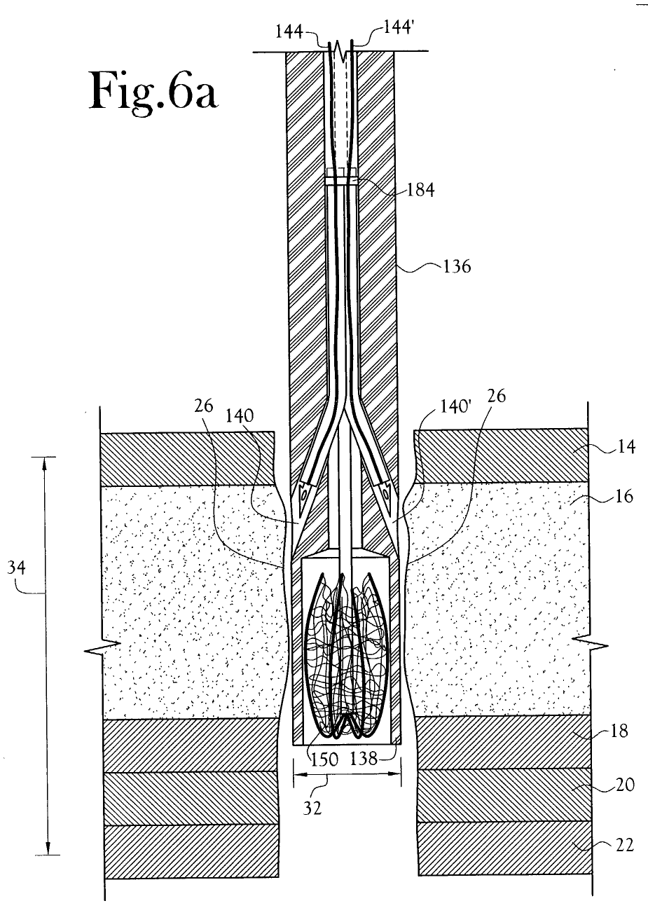

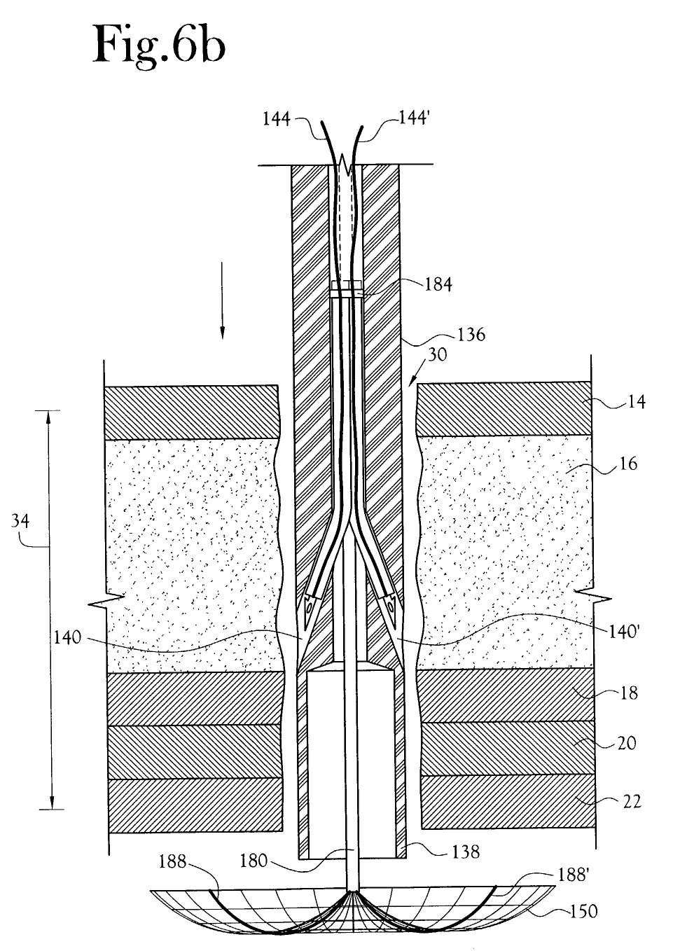

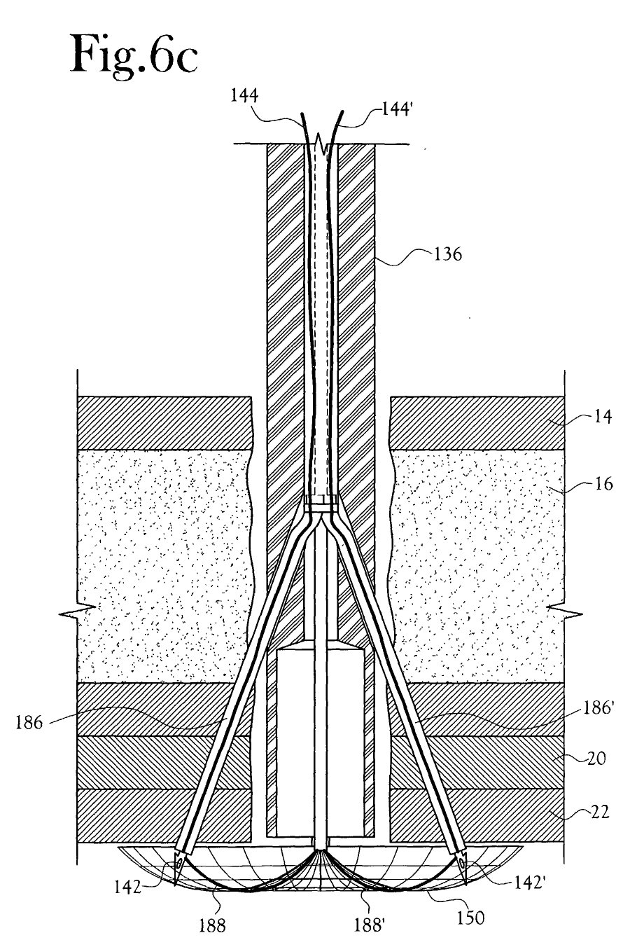

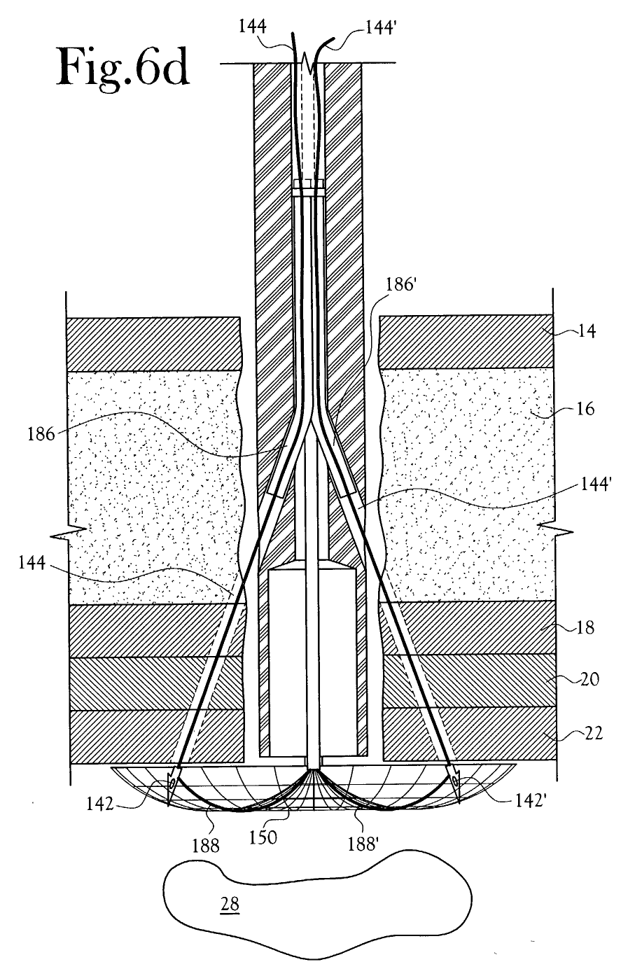

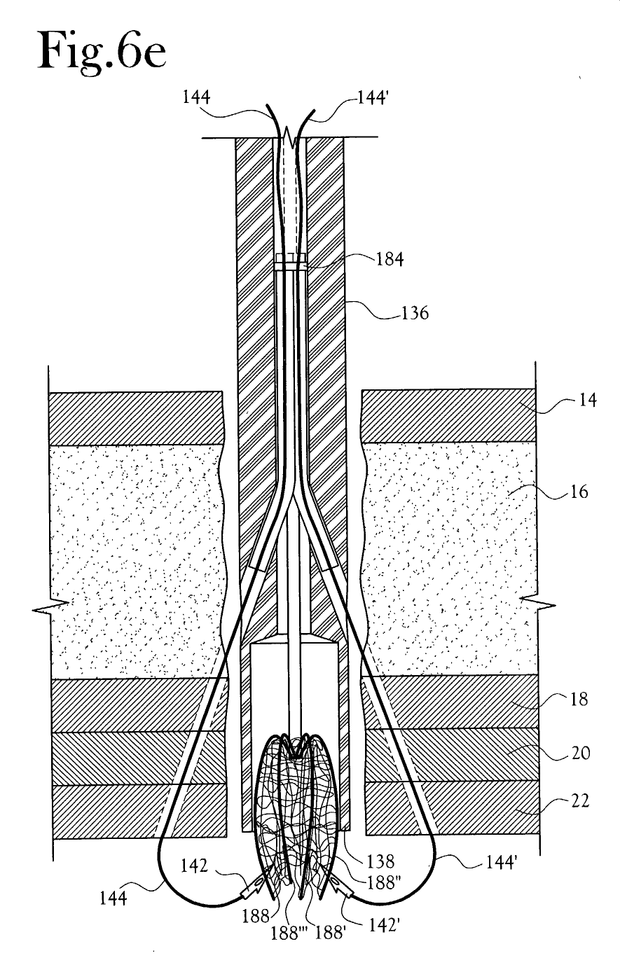

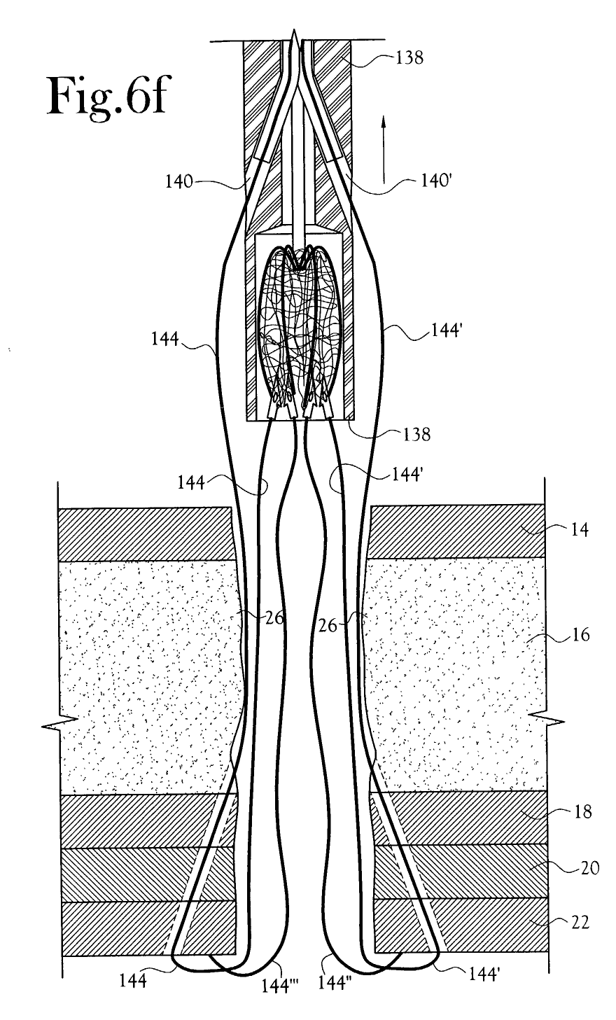

The incision closing apparatus 110 includes an elongated insertion probe 136 having an elongated tube configured in width to insert through a generally narrow incision width 32. A distal end of the insertion probe 136 forms an insertion end 138 that is sized in length and shaped to be removably insertable an adequate depth 34 through the dermal layer 14, the fat layer 16, and the fascia layer 18 of the patient. Upon positioning of the insertion end 138 at an adequate depth 34 for the distal end to be disposed interior of the fascia layer 18, an operator manipulates appropriate controls for extending a shield 150 from the insertion end 138, and for ejection and placement of a plurality of suture needles 142, 142′, 142″, 142′″ (see FIGS. 4-8), through the sides and layers of the incision through the fascia layer 18 and adjacent tissue layers 20, 22. The shield 150 protects internal organs 28 from being penetrating by the ejected needles 142, 142′, 142″, 142′″. The incision closing apparatus 110 includes at least two openings 140, 140′ (see FIG. 4) in opposed orientation along the circumference of the insertion end 138. A preferred embodiment includes four openings 140, 140′, 140″, 140′″, disposed in spaced-apart orientation along the circumference of the insertion end 138. The openings 140, 140′, 140″, 140′″ are generally aligned at a selected distance from the distal end of the insertion end 138. The selected distance from the distal end is approximately the combined width of the fascia layer 18, the muscle layer 20, and the peritoneum layer 22, in order to position each opening proximal to the fascia layer 18 for proper placement of the needles during ejection of the suture needles 142, 142′, 142″, 142′″ through the fascia layer 18 (see FIG. 6c).

The insertion probe 136 is removably connected to a control housing 172 at a connector end 172″. The control housing 172 may be shaped in the form of a gripping means such as a handle at a first end 172′ (see FIG. 4). The probe insertion end 138 is sized to include a sufficient length to provide a spaced apart distance from the connector end 172″ to allow an operator to work a comfortable distance above and/or to the side of the patient during the incision closing procedure. A sufficient length of the insertion probe 136 may include a length in the range of about six inches to about twelve inches from the connector end 172″ to the insertion end 138. The overall length of the incision closing apparatus 110 may include a length in the range of about fourteen inches to about eighteen inches from the handle end 172′ to the insertion end 138. After completion of the surgical procedure utilizing the insertion probe 136, the incision closing apparatus 110 may be discarded and a sterile dermal incision closing apparatus (not shown) may be provided for closure of the dermal incision. Alternatively, the insertion probe 136 may be detached at the connector end 172″, discarded, and a replacement insertion probe 136 having a sterile insertion end 138 may be attached to the control housing 172. Alternatively, after use of the insertion probe 136, the used plurality of suture needles 142, 142′, 142″, 142′″ are discarded and replacement needles may be reinserted into respective channels adjacent each opening of the insertion end 138, after connection of a suture filament 144 to each needle for use after sterilization of the incision closing apparatus 110.

Positioned on the control housing 172 is a control means including two or more actuator buttons, switches, or slide members 174, 176. A first slide member 174 actuates the movement of a first mechanical connector 180 that activates the opening of the shield 150 extendable from the insertion end 138. A second slide member 176 actuates a needle ejection means including a second mechanical connector 182 extended longitudinally through the interior 146 of the insertion end 138. The control means includes the first and second slide members 174, 176 being manipulated by the operator along two tracks 178, 178′ along the control housing 172, thereby providing operator control of the timing and rate of extension and retracting of the shield 150, and the timing and rate of extension of the plurality of needles 142, 142′, 142″, 142′″ from the insertion end 138. The slide members 174, 176 are connected by mechanical linkages to the first mechanical connector 180 and second mechanical connector 182. The mechanical linkages include generally a rigid wire or a segmented rod member serving as a first mechanical connector 180, or an internal tube member serving as a second mechanical connector 182 that extend internally along the lengthwise axis through the insertion probe 136 for connection to a shield actuator assembly and a needle actuator assembly in insertion end 138 (see FIGS. 4-5).

One embodiment of the extending and retracting mechanism for the shield 150 includes the first mechanical connector 180 connected at a distal end to a first or base end of a plurality of flexible spokes 188, 188′, 188″, 188′″ that are disposed in bent positions within the distal, open end of the insertion end 138. The flexible spokes 188, 188′, 188″, 188′″ include second ends attached to respective quadrants of one side of the shield 150. The shield 150 is composed of a pliable material such as cloth, a plastic mesh, or a metal mesh. The shield 150 of pliable material is unfolded into an extended position as the flexible spokes 188, 188′, 188″, 188′″ extend upon being pushed out of the distal end of the insertion end 138. When the operator manipulates slide member 174, the first mechanical connector 180 extends downwards through the insertion probe 136 to exert force against the base ends of each flexible spoke 188, 188′, 188″, 188′″ for extension outwards and unfolding of the shield 150 to an extended position (see FIGS. 5 and 6b). For closure of the shield 150 after ejection of the needles 142, 142′, 142″, 142′″, a reverse of the opening process occurs except that the shield 150 is inverted by being pulled backwards into the distal end of the insertion end 138 with the plurality of needles 142, 142′, 142″, 142′″ imbedded into one side of the shield 150.

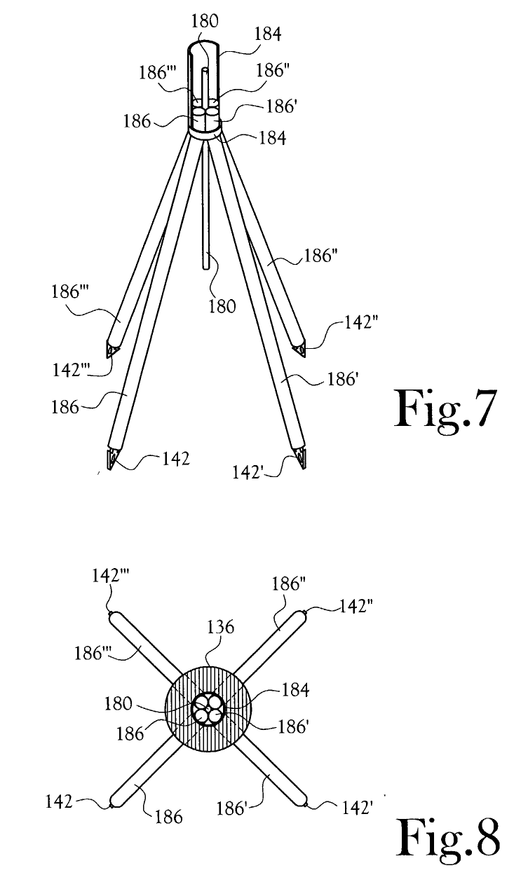

Activation of the ejection of each respective suture needle 142, 142′, 142″, 142′″ through and outwardly from each respective opening 140, 140′, 140″, 140′″ includes an ejection assembly connected to a distal end of the tube member of the second mechanical connector 182 disposed within the insertion end 138 and proximal to the openings 140, 140′, 140″, 140′″. The ejection assembly includes the tube member having a ring member 184, and a plurality of elongated needle spokes 186 that are separately connected to the ring member 184 in a radial pattern at a base end of each needle spoke 186. Each needle spoke 186, 186′, 186″, 186′″ is elongated in length (see FIGS. 7-8) and is composed of a material having a spring element that is biased to extend each suture needle outwardly through each channel when pushed by the tube member of the second mechanical connector 182 and the ring member 184 toward the openings 140, 140′, 140″, 140′″. Each respective suture needle 142, 142′, 14″, 142′″ is releasably attached to a distal end of each elongated needle extending spoke 186, 186′, 186″, 186′″. As the ring member 184 is extended downwardly toward the insertion end 138 by movement of the tube member of the second mechanical connector 182, the base end of each needle extending spoke 186, 186′, 186″, 186′″ is pushed outwardly through each angled channel leading to each opening 140, 140′, 140″, 140′″. By extending each spoke 186, 186′, 186″, 186′″ through respective channels and openings 140, 140′, 140″, 140′″, each respective needle 142, 142′, 142″, 142′″ is ejected from each opening of the insertion end 138 in a generally downwardly and outwardly angled direction for insertion through opposed sides of the incision through the fascia layer 18, and through any adjacent tissue layers 20, 22 (see FIG. 6c). When the shield 150 is in the extended position, the needles 142, 142′, 142″, 142′″ are imbedded into the shield 150 with the suture filaments 144, 144′, 144″, 144′″ trailing from each imbedded needle 142, 142′, 142″, 142′″ (see FIG. 6d).

For retraction of each ejected needle extending spoke 186, 186′, 186″, 186′″ back into the internal, angled channels of openings 140, 140′, 140″, 140′″ after imbedding of each needle 142, 142′, 142″, 142′″ into the shield 150, the operator manipulates the slide member 176 from a second position to a first position, with associated retracting of tube member of the second mechanical connector 182, and resulting pulling of the ring member 184 upwards within the insertion end 138 (see FIG. 6e). Each needle spoke 186, 186′, 186″, 186′″, without a needle, is retrieved into each opening 140, 140′, 140″, 140′″ proximal of the insertion end 138. Each suture filament 144, 144′, 144″, 144′″ remains inserted through the respective fascia layer 18 and adjacent tissue layers 20, 22, with each suture filament extending back into openings 140, 140′, 140″, 140′″.

In an alternative embodiment of the insertion probe 136, the extendable shield 150 includes at least two shield portions disposed within the insertion end 138 in a first, closed position. The respective shield portions are ejected out of the insertion end 138 for lateral opening as a hemispherical, porous shield 150 due to the extension of spring members such as a plurality of spring-tensioned spokes 188 in a manner similar to unfolding of an umbrella, when the shield slide member 174 is triggered by an operator. The shield 150 is disposed proximal to the exterior surface of the insertion end 138 by manipulation of the shield slide member 174 that actuates the first mechanical connector 180 for pushing the shield 150 out of the distal end of the insertion end 138 and into the open, unfolded position (see FIG. 6b). Upon opening the shield 150 within the patient, the shield 150 may be repositioned so that each shield portion of shield 150 is laterally disposed on either side of an incision through the fascia layer 18 and an adjacent, inner tissue layer, such as the peritoneum 22. The shield 150 is retractable into the insertion end 138, by a reverse folding of the shield 150 into the insertion end 138 in a retracting position 190 similar to an umbrella turning inside-out. The retraction of the respective shield portions of the shield 150 into the insertion end 138 is accomplished by an operator's re-manipulation of the shield slide member 174 on the control housing 172 and the retraction of first mechanical connector 180 toward the handle end 178′ (see FIG. 6e). After retraction of the shield 150, the insertion end 138 is removed from the incision by the operator (see FIG. 6f).

Within the control housing 172, a supply of suture thread and/or suture filaments are stored on a reel means such as a spool or reel device 148 disposed within an enclosure 148′. An alternative housing (not shown) may include a “quick-connect” opening into which a cassette or cartridge is attached for providing a source of suture threads or filaments. The reel device 148 may include at least one spring tensioning means 192 having at least one tensioning spring connected to the reel device 148 that allows extension of a plurality of continuous suture threads or filaments 144, 144′, 144″, 144′″ through the generally hollow interior 146 of the insertion probe 136 when suture needles 142, 142′, 142″, 142′″ are ejected from an insertion end 138. The insertion end 138 includes an external side surface through which at least two openings 140, 140′ are disposed (see FIG. 4). The at least two openings 140, 140′ are preferably positioned on opposed sides of the external surface of the insertion end 138 to allow ejection of suture needles 142, 142′, 142″, 142′″ into and through opposed sides and/or layers of an incision through the fascia layer 18 and adjacent tissue layers 20, 22.

During insertion of the insertion end 138 into an incision into the patient, the shield 150 is maintained in a folded position within the interior of the distal portion of the insertion end 138 to allow rapid insertion through the respective incisions in the dermal layer 14, through the layer of fat 16, and through the incision in the fascia layer 18. After the insertion end 138 is inserted past the fascia layer 18, the muscle layer 20, and/or the peritoneum 22 (see FIG. 6b), the shield 150 is extended outwardly into an unfolded, lateral position from the insertion end 138. The shield is positioned for receipt of each needle 142, 142′, 142″, 142′″ ejected from respective angled channels and openings 140, 140′, 140″, 140′″ after the needles are extended through the adjacent sides of the incision through the fascia 18 and adjacent tissue layers 20, 22 (see FIG. 6c). The operator of the insertion probe 136 re-positions the insertion end 138 and the shield 150 to a third position against the interior side of the fascia layer 18, the muscle layer 20, and/or the peritoneum 22, thereby providing a temporary anchor for the insertion end 138 during a step of simultaneously ejecting needles 142, 142′, 142″, 142′″ through respective side walls and/or layers of the incision through the fascia layer 18, and adjacent tissue layers 20, 22. When the insertion end 138 is repositioned in the third position, the shield 150 is extended at about an angle of about 90 degrees from the first mechanical connector 180 within the insertion end 138. As illustrated in FIGS. 6c and 6d, the extended shield 150 serves to provide a surface into which the ejected needles 142, 142′, 142″, 142′″ are imbedded, and serves to retain the needles 142, 142′, 142″, 142′″ from penetrating past the fascia layer 18 and into the abdominal cavity 24, thereby protecting against needle penetration into an organ 28. Due to the limited width 32 of the incision in the fascia layer 18, and the shifting 26 of the fat layer 16 inwardly into the channel formed by the incision through the dermal layer 14 and the fascia layer 18, an operator may not be able to visually verify whether one or more of the ejected needles 142, 142′, 142″, 142′″ have been inserted into the target tissue layers, or have penetrated into the abdominal cavity 24 and into an organ 28. Rapid retraction of the insertion end 138 is a benefit to allow the operator to inspect the interior placement of the needles 142, 142′, 142″, 142′″. The inwardly folding of the shield 150 into the insertion end 138 (see FIG. 6e), provides a retraction step of collecting each of the two or four ejected needles 142, 142′, 142″, 142′″ within the collapsed shield 150 without the risk of a needle being left within the patient. During removal of the insertion end 138 from the incision in the patient, the suture filaments 144, 144′, 144″, 144′″ are retained internally (see FIGS. 6e and 6f), and remain extended through opposed sides of the incision through the internal fascia layer 18, and adjacent tissue layers 20, 22. The retracted insertion probe 136 retains within the insertion end 138, each of the needles 142, 142′, 142″, 142′″ imbedded in the shield 150, thereby providing an incision closing apparatus 110 that is disposable after the plurality of suture filaments 144, 144′, 144″, 144′″ are collected by the operator.

31 After implanting the filaments 144, 144′, 144″, 144′″ through opposed side walls and/or layers of the incision through the fascia layer 18, the operator may manipulate inwardly the insertion end 138 further into the abdominal cavity 24 to a fourth position (not shown), for closure of the shield 150 within the insertion end 138 by the operator's manipulation of actuator button 174. During the step of removing the insertion end 138 (see FIG. 6f) from the patient, the respective suture filaments 144, 144′, 144″, 144′″ remain extended from the respective openings 140, 140′, 140″, 140′″. After removal of the insertion end 138 from the patient, the suture filaments 144, 144′, 144″, 144′″ are tied for closure of the incision in the fascia layer 18, followed by insertion of dermal stitches or closure of the dermal incision by a dermal incision closure method to complete the laparoscopic incision closure procedure.

An alternative embodiment may include an internal incision closing apparatus having an insertion probe providing three or two openings on opposed side portions of the insertion end, with each opening having at least one needle and one or more means for ejecting positioned therein. As provided in detail above, the ejection means includes at least one elongated needle spoke having at least one needle removably disposed thereon, and the needle having at least one suture filament connected thereto. An alternative needle spoke may include two needles positioned on a distal end of each needle spoke, with separate suture filaments connected to each respective needle. An additional embodiment may include one generally elongated and horizontally oriented opening (not shown) that is disposed on a side portion of an insertion end for ejection of at least two needles therefrom. The insertion end is sized in length to position each opening, whether a single opening or more than one opening, adjacent to the fascia layer within the patient. A second horizontal opening (not shown) may be disposed adjacent to, or above, the first opening if additional needles are to be ejected at differing angles and/or heights along the insertion probe. In each alternative embodiment, a flexible shield (see FIGS. 4, 5 and 6b) is extendable from an open end of the insertion probe. The shield is disposed from a retracted position within the insertion end to an extended position by an extending mechanism within the insertion end and a plurality of shield extending spokes attached to one side of the shield. The single opening is positioned on a side portion of the insertion end, at about a spaced-apart distance from the distal end so that the opening is disposed adjacent the sides of the incision through the fascia layer when the shield is opened into an extended position interior of the fascia layer in the patient. At least two needles, each having suture filaments connected thereto, are extended from the first opening by an ejection means as illustrated in FIGS. 4-8. Alternative embodiments utilizing an insertion probe including a combination of features as illustrated in FIGS. 4-8 may be utilized for simultaneous ejection and insertion of a plurality of suture needles, each having at least one suture filament attached thereto, through adjacent sides of an incision through one or more internal tissue layers, with each suture needle being imbedded into a shield opened in an extended position, for rapid closure of an internal incision without harming of an internal organs in the patient.

A method for closure of internal incisions in a fascia layer 18 interior of a patient is disclosed, utilizing a laparoscopic incision closing apparatus 110. The method for closure includes a step of providing a insertion probe 136 having the elements illustrated in FIGS. 4-5, as described herein. The insertion probe 136 includes an insertion end 138 having an extendable shield 150 that is maintained in a retracted, first position within the insertion end 138 (see FIG. 6a), until the insertion end 138 is properly positioned within an incision through a fascia layer 18. A step of inserting includes positioning the insertion end 138 of the insertion probe 136 through a dermal incision 30 in the patient by an operator's guiding the insertion end 138 through the dermal layer 14, and further inserting the insertion end 138 to a first position (see FIG. 6a) through an incision in the fascia layer 18 within the patient. The step of inserting and positioning the insertion end 138 to a first position interior of the fascia layer 18 is followed by a step of extending outwardly of the shield 150, including one continuous shield portion or, alternatively, two shield portions that are laterally extended from the distal portion of the insertion end 138 (see FIG. 6b). Upon extending of the shield 150, the insertion end 138 is repositioned to a second position (see FIG. 6c), with the shield 150 positioned against an interior surface of the fascia layer 18 and any intervening layers 20 and/or 22. The first step of repositioning is followed by a step of ejecting at least two, and preferably four needle spokes 186, 186′, 186″, 186′″ having a suture needle 142, 142′, 142″, 142′″ on each needle spoke, through each respective opening 140, 140′, 140″, 140′″ at about the same time (see FIG. 6dc from the spaced apart holes in the insertion end 138. The ejecting step directs at least two needles, and preferably four needles, through respective adjacent sides and/or layers of the incision in the fascia layer 18, with each inserted needle 142, 142′, 142″, 142′″ having at least one suture filament 144, 144′, 144″, 144′″ extending therefrom, and with each suture filament 144, 144′, 144″, 144′″ extending backwards to each respective opening 140, 140′, 140″, 140′″ in the insertion end 138. After ejecting and implanting each needle 142, 142′, 142″, 142′″ into the shield 150, a step of retracting of the needle spokes 186, 186′, 186″, 186′″ occurs (see FIG. 6d), before a step of repositioning moves the insertion end 138 interior of the fascia layer 18 to a third position (see FIG. 6e), for closure of the shield 150 into the insertion end 138. The step of repositioning is followed by a step of removing the insertion end 138 from the incision in the fascia layer 18 and from the dermal incision 30, with two or more suture filaments 144, 144′, 144″, 144′″ trailing from the insertion end 138 while remaining inserted through the side portions of the fascia layer incision 18 and adjacent tissue layers 20, 22. After the step of removing the insertion end 138, an additional step of tying the plurality of suture filaments 144, 144′, 144″, 144′″ is completed by the surgeon, thereby closing the incision in the fascia layer 18 within the patient. The step of tying includes separating the suture filaments from the shield 150 of the laparoscopic incision closing device 110 for completion of the step of tying of the suture filaments 144, 144′, 144″, 144′″ in order to complete the closure of an incision through one or more internal tissue layers within the patient. The surgeon may then insert dermal incision stitches for closure of the dermal incision 30, thereby completing the laparoscopic incision closure procedure. The method for closure utilizing the laparoscopic incision closing apparatus 110 provides an efficient suture technique for closing an internal incision through a fascia layer 18 and/or through adjacent internal tissue layers 20, 22, while protecting interior organs 28 from being punctured by ejected needles 142, 142′, 142″, 142′″.

While the present invention has been illustrated by description of several embodiments and while the illustrative embodiments have been described in considerable detail, it is not the intention of the applicants to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described herein. Accordingly, departures may be made from such details without departing from the spirit or scope of the applicants general inventive concept as described in the appended claims.

Having thus described the aforementioned invention, we claim:

1. A medical apparatus for closure of an incision through an internal tissue layer within a patient, comprising:
a housing sized to be manipulated by an operator, said housing having a control means thereon, said housing having a supply of suture filaments dispensed from said housing;
an insertion member connected to said housing, said insertion member including an insertion end having a plurality of openings disposed a spaced apart distance from a distal end of said insertion end, said insertion end is sized for insertion through a dermal layer incision and for insertion through an incision in an internal tissue layer to position said plurality of openings adjacent the incision;
a shield disposed in a folded configuration within said insertion end, said shield is retractably extended from said insertion end by a first mechanical connector disposed between said control means and said insertion end, said first mechanical connector is connected to said shield for extension of said shield to an extended position by manipulation of said control means upon insertion of said insertion end interior of the incision in the internal tissue layer, said shield is retractably folded into said insertion end by re-manipulation of said control means; and
a plurality of suture needles disposed within said insertion end for ejection of each suture needle from each one of said plurality of openings, each suture needle having a suture filament connected thereto, each suture filament is extended through said insertion member to said supply of suture filaments in said housing, each suture needle is attached in an ejection assembly connected by a second mechanical connector to said control means, each one of said plurality of suture needles are ejected distally from respective openings toward said insertion end by said ejection assembly actuated by said second mechanical connector upon the operator's manipulation of said control means, for insertion of each suture needle and suture filament through opposed sides of the incision in the internal tissue layer, whereby each suture needle is imbedded in said shield disposed in said extended position within the patient.

2. The medical apparatus of claim 1 wherein said shield is composed of a porous material and is disposed laterally to encircle said insertion end in said extended position, said porous material having a mesh configuration supported by a plurality of flexible extension spokes, said mesh configuration provides for retention of said suture needles thereon.

3. The medical apparatus of claim 2 wherein said plurality of openings are oriented a spaced apart distance laterally on said insertion end, each opening having an angled channel therein through which each respective suture needle is simultaneously ejected outwardly from each angled channel by said ejection assembly for insertion of each suture needle through respective sides of the incision in the internal tissue layer.

4. The medical apparatus of claim 3 wherein said ejection assembly includes:
an actuator assembly having a plurality of needle spokes disposed in a radial pattern within said insertion end proximal to said openings, each needle spoke is releasably attached to each respective suture needle;
a ring member attached at a base end of each respective needle spoke, said ring member is connected to said first mechanical connector; and
said second mechanical connector is extended along a lengthwise axis of said insertion member between said control means and said ring member;
whereby when the operator manipulates said control means, said second mechanical connector moves toward said insertion end, said ring member is moved toward said insertion end resulting in said needle spokes being extended through said respective openings.

5. The medical apparatus of claim 4 wherein each needle spoke is releasably attached to each respective suture needle during ejection of each suture needle from respective openings and during imbedding is said shield, said needle spokes are retracted into respective openings upon re-manipulation of said second mechanical connector with each suture needle having said suture filament attached thereto remaining imbedded in said shield.

6. The medical apparatus of claim 5 wherein said housing including:
said control means includes a first and a second actuator slidably disposed on said housing, said first and second actuators are connected respectively to said first and second mechanical connectors, said first and second actuators are manipulated by the operator between first and second positions for respective extension of said shield and ejection of said plurality of suture needles; and
a reel device having suture filaments disposed thereon, said reel device includes a plurality of suture filaments extendable from said reel device, through said insertion end and through said plurality of openings for attachment to respective suture needles, each suture needle and attached suture filament is ejected into respective sides of the incision in the internal tissue layer.

7. The medical apparatus of claim 2, wherein each of said plurality of flexible extension spokes having a spring tension biased to extended outwardly from said insertion end when said first mechanical connector is extended through said insertion end.

8. The medical apparatus of claim 7 wherein said plurality of flexible extension spokes having said spring tension are radially along one side of said shield, each of said flexible extension spokes extend to distal ends disposed in spaced-apart orientation proximal to a perimeter of said shield, said flexible extension spokes are collapsible into said insertion end upon manipulation by the operator of said control means for retraction of said first mechanical connector through said insertion end and toward said housing.

9. An incision closing apparatus for closure of an incision through a fascia layer within a patient, comprising:
an insertion member including a housing having a control means thereon and having a supply of suture filaments disposed to dispense through said housing, said insertion member having an insertion end extended a selected length from said housing for insertion of said insertion end through an incision through a dermal layer and a fascia layer within a patient;
a plurality of openings disposed in spaced apart orientation along a peripheral surface of said insertion end, each of said plurality of openings aligned with one of a plurality of channels angled to extend within said insertion end;
a shield extendable from said insertion end, said shield is retractably extended from said insertion end by extension of a first mechanical connector disposed to extend between said control means and said shield within said insertion end, said shield is expanded to an extended position laterally oriented around said insertion end by the operator's manipulation of said control means upon insertion of said insertion end through the incision and distal of the fascia layer, said shield is retractably inverted into said insertion end by re-manipulation of said control means for removal of said insertion end from the incision through the fascia layer; and
a plurality of suture needles positioned individually in each one of said channels angled to extend within said insertion end, each suture needle is simultaneously ejected from each respective opening by a second mechanical connector disposed to connect to each suture needle within said insertion end, said suture needles are simultaneously ejected by said second mechanical connector from each opening upon the operator's manipulation of said control means;
whereby upon ejection of each suture needle from each opening, said suture needles are inserted through respective sides of the incision through the fascia layer and are imbedded into said shield in said extended position disposed distal of the fascia layer within the patient.

10. The incision closing apparatus of claim 9 wherein each of said suture needles include at least one suture filament attached thereto, each suture filament is extended from each suture needle and through said insertion member for connection to said supply of suture filaments, whereby upon said suture needles imbedded into said shield in said extended position, each suture filament extends from each respective opening to each respective suture needle for extension through respective sides of the incision through the fascia layer within the patient.

11. The incision closing apparatus of claim 10 wherein said second mechanical connector includes:
a plurality of elongated spokes having a distal end on which one suture needle is disposed, each elongated spoke having a base end disposed within said injection end;
a ring member encircles said base end of each elongated spoke, said ring member in mechanical communication by an internal tube member disposed along the lengthwise axis of said insertion member, said internal tube member and said ring member are moved distally along the lengthwise axis of said insertion member upon the operator's actuation of said control means on said housing;
said plurality of channels angled within said insertion end are disposed in a radial orientation within said insertion end, each channel is angled toward said insertion end for direction of each elongated spoke having a needle thereon on a path angled outwardly and distally of each respective opening;
each of said elongated spokes are retrievably extended from each respective opening for insertion of each suture needle through respective sides of the incision through the fascia layer, whereby each suture needle is imbedded into said shield in said extended position.

12. The incision closing apparatus of claim 11 wherein said shield having a base end connected to said first mechanical connector, said shield base end is extended to said extended position laterally of said insertion end, whereby said shield in said extended position is positioned interior of the fascia layer for imbedding of said suture needles into said shield.

13. The closing apparatus of claim 12 wherein said control means including an actuator control device having a plurality of buttons for actuation by the operator, said plurality of buttons including a first and a second slide member in mechanical connection respectively with said first and second mechanical connectors for extension of said shield and for movement of said ring member for extension of said elongated spokes having suture needles thereon.

14. An incision closure apparatus for insertion of suture needles and suture filament for closure of an incision through an internal tissue layer within a patient, comprising:
an insertion member including a housing having a control means thereon, said insertion member having an insertion end extended a selected length from said housing, said insertion end having a plurality of openings in spaced apart orientated laterally along said insertion end, said insertion end is sized to be removably inserted through a dermal incision substantially aligned with an incision through an internal tissue layer within a patient;
a shield extendable from an interior of said insertion end, said shield including a plurality of spring members connected radially to said shield, said plurality of spring members are biased to extend said shield to an extended position from said insertion end upon actuation of the operator of said control means in communication with a first mechanical connector, said shield in said extended position is disposed laterally from said insertion end and is disposed distally of the internal tissue layer within the patient, said shield is inverted to a closed position within said insertion end by re-actuation of said control means by the operator; and
a plurality of needles disposed within said insertion end, each needle having a suture filament attached thereto, each needle is extended by a respective needle spoke mechanism connected to a second mechanical connector for substantially simultaneous ejection from respective openings when said shield is extended to said extended position;
whereby upon ejection of each needle from each respective opening, each needle is inserted through respective sides of the incision through the internal tissue layer, said suture filament from each needle is extended from each respective opening and through respective sides of the incision through the internal tissue layer within the patient.

15. The incision closure apparatus of claim 14 wherein said shield is maintained in said extended position by said plurality of spring members for positioning against an interior surface of the internal tissue layer, said needle spoke mechanism ejects each respective needle through respective sides of the internal incision for imbedding into said shield maintained proximal to the internal tissue layer, whereby an internal organ within the patient's is not imbedded with any one of said needles.

16. A method for closure of an interior incision through an internal tissue layer within a patient, comprising the steps of:
providing an incision closing device including an insertion end having spaced apart lateral openings therein, said insertion end having a plurality of needles disposed for simultaneously ejecting from respective lateral openings;
inserting said incision closing device through an incision in a dermal layer and through an interior incision in an internal tissue layer;
positioning said insertion end interior of the internal tissue layer;
extending a shield distally from said insertion end into a laterally extended position;
repositioning said insertion end and said extended shield against an interior side of the internal tissue layer;
ejecting a plurality of needles having suture filament attached thereto from said spaced apart openings, said ejecting step including inserting each needle through opposed sides of the interior incision in the internal tissue layer;
imbedding each needle into respective surfaces of said shield;
repositioning said insertion end interior of the internal tissue layer;
collapsing said shield into said insertion end, said shield retaining said needles imbedded therein; and
removing said insertion end from the incision, said needles imbedded in said shield having suture filaments trailing therefrom for an operator to collect the respective suture filaments for closure of the incision in the internal tissue layer within the patient.

17. The method for closure of claim 16 wherein said step of ejecting includes ejecting each of said plurality of needles having suture filament attached thereto, whereby upon inserting each of said plurality of needles having suture filaments attached through respective sides of the interior incision in the internal tissue layer in the patient, said suture filaments extend through each opposed side of the interior incision.

18. The method for closure of claim 17 including a step of closing of the interior incision by tying respective suture filaments together, said step of tying including detachment of each respective suture needle from respective suture filaments, whereby said step of closing providing closure of the internal incision in the internal tissue layer within the patient without a need for visual observation of the closure.

19. An incision closure apparatus for insertion of a plurality of needles having suture filaments attached thereto for closure of an interior incision through a fascia layer and adjacent tissue layers within a patient, comprising:
an insertion member including a housing having a control means thereon, said insertion member having an insertion end extended a selected length from said housing, said insertion end having at least two openings oriented laterally along said insertion end, said insertion end is sized to be removably inserted through a dermal incision substantially aligned with an internal incision through a fascia layer and adjacent tissue layers within a patient;
a shield extendable from said insertion end into an extended position of a lateral orientation relative to said insertion end by a plurality of spring members supporting said shield, said shield is extended from said insertion end by a first mechanical connector actuated by manipulation of said control means upon the operator's insertion of said insertion end distal of the fascia layer and adjacent tissue layers within the patient, said shield is inverted into a closed position within said insertion end by the operator's re-actuation of said control means; and
a needle disposed in each respective opening within said insertion end, each needle having a suture filament attached thereto, each needle is positioned in an outwardly angled orientation by an elongated needle spoke disposed to be ejected by a second mechanical connector from each respective opening within said insertion end, said second mechanical connector simultaneously ejects each needle from respective openings when said shield is extended to said extended position and said control means is actuated;
whereby upon ejection of each needle from said opening, said plurality of needles are inserted through respective sides of the interior incision through the fascia layer and adjacent tissue layers, said suture filament from each needle is extended through respective sides of the interior incision through the fascia layer and adjacent tissue layers within the patient.

20. The incision closure apparatus of claim 19 wherein said first and second mechanical connectors including:
said first mechanical connector is extended along an axis of said insertion end, said first mechanical connector disposes said shield into said extended position against an interior of the fascia layer upon the operator's manipulation of said control means;
said second mechanical connector is extended along the axis of said insertion end, said second mechanical connector provides for simultaneous ejection of each one of said plurality of needles from respective openings upon the operator's manipulation of said control means, each needle is directed from each opening for angled ejection through opposed sides of the incision through the fascia layer and adjacent tissue layers, each needle becomes imbedded into said shield in said extended position; and
said first mechanical connector disposes said shield into said extended position with angled ejection of said plurality of needles upon the operator's actuation of said control means;
whereby said shield is maintained in said extended position against the interior of the fascia layer and adjacent tissue layers for capture of each ejected needle through opposed sides of the incision for imbedding into said shield, thereby an internal organ proximal to the fascia layer and adjacent tissue layers is not imbedded with said needles.
